(19) United States
(12) Patent Application Publication
(10) Pub. No.: US 2014/0286868 A1
(43) Pub. Date: Sep. 25, 2014

(54) IMMORTALIZED HUMAN PROSTATE CELL LINES

(71) Applicant: Tuskegee University, Tuskegee, AL (US)

(72) Inventors: Clayton Yates, Auburn, AL (US); Timothy Turner, Auburn, AL (US)

(73) Assignee: TUSKEGEE UNIVERSITY, Tuskegee, AL (US)

(21) Appl. No.: 14/296,676

(22) Filed: Jun. 5, 2014

Related U.S. Application Data
(63) Continuation of application No. 13/589,809, filed on Aug. 20, 2012, now abandoned.
(60) Provisional application No. 61/525,570, filed on Aug. 19, 2011.

(51) Int. Cl. A61K 49/00; G01N 33/50

(52) U.S. Cl. CPC A61K 49/0008; G01N 33/5011; G01N 2500/04; G01N 2500/10
USPC 424/9.2; 435/371; 800/10; 435/39

(57) ABSTRACT

Immortalized human cell lines derived from prostate cells are disclosed. Immortalized cells derived from a human epithelial prostate tissue cancer tumor are provided, as well as immortalized cells derived from healthy human epithelial prostate tissue from the same patient. Methods for utilizing such immortalized cell lines for researching, screening, and evaluating antimalignancy therapies and drug candidates are also disclosed.

### RELATED APPLICATIONS

The application claims priority to US provisional application 61/525,570 (filed on Aug. 19, 2011) and 61/467,357 (filed on Mar. 24, 2011), both of which are herein incorporated by reference.

### FIELD OF THE INVENTION

The present invention relates to immortalized human prostate cell lines derived from prostate cancer tissue, and methods and uses thereof in the research of prostate cancer. More particularly, the present invention relates to paired (first and second) immortalized human prostate cell lines derived from a single human patient, where a first cell line is derived from prostate cancer tissue of the patient and a second cell line is derived from healthy prostate tissue of the patient, and methods and uses thereof in the research of prostate cancer.

### BACKGROUND OF THE INVENTION

In the United States, prostate cancer is currently the most commonly diagnosed cancer and the second-leading cause of cancer death in men. Following trends of increasing and decreasing rates, prostate cancer rates have been, since 2001, decreasing by 4.4% per year. Likewise, since the early 1990s, there has been a more substantial decrease in prostate cancer deaths among African American men than their white counterparts. Furthermore, African American men and Jamaican men of African descent have the highest incidences of prostate cancer in the world. African American men have a 60% higher incidence and mortality rates from prostate cancer compared to Caucasian men in North America, indicating that prostate cancer is a major public health problem in populations of African descent. The etiology of these racial differences in the clinical manifestation of prostate cancer is unclear, however hormonal, genetic, behavioral and environmental factors have all been implicated by various studies (see Moul, J W, Screening for prostate cancer in African Americans, Curr. Urol. Rep. 1: 57-64, 2000). Many factors have been suggested to contribute to the higher prostate cancer incidence and mortality rates in African American men. For example, over-expression of the androgen receptor (“AR”) and elevated luteinizing hormone (“LH”) levels have been associated with advanced disease progression. Although substantial efforts have been applied to identify agents with efficacy against prostate cancer, few treatment options, including classical chemotherapeutic agents, have not proven to be effective against this disease.

Early stage prostate cancers are androgen dependent, and thus can be treated in part via hormone therapy. Most hormone dependent cancers become refractory (i.e., hormone independent) after one to three years and resume growth despite hormone therapy. Hormone-refractory prostate cancer is a late stage of prostate cancer for which better treatments are needed. Thus, therapies directed at preventing or limiting the tumor's transition to the more aggressive invasive and metastatic stages offer benefits different from the present therapies that were designed to kill prostate cancer cells.

To understand the many factors suspected of contributing to the development of this malignancy, there is a critical need for in vitro models representing primary tumors. For many years, efforts have been made to develop immortalized human cell lines adapted to the study of specific human diseases, such as cancers. However, no suitable in vitro models which accurately reflect the in situ characteristics of malignant epithelium for the study of African American prostate cancer are available. In particular, research into molecular and genetic mechanisms underlying prostate carcinogenesis in high-risk African American men would be greatly advanced by in vitro models of African American prostate tumors representing primary tumors.

It is possible to immortalize normal cells, that is to say make them capable of multiplying indefinitely. Indeed, normal cells do not survive more than a ten passages. For that, techniques for the transfection of cells, with the aid of specially adapted vectors, such as the SV40 vector comprising a sequence of the large T antigen (R. D. Berry et al., Br. J. Cancer, 57, 287-289, 1988), or a vector comprising DNA sequences of the human papillomavirus (U.S. Pat. No. 5,376,542), are generally used.

Among the cells often involved in the onset of cancer are the epithelial cells. Epithelial cells differ from other cells of the human body in the expression of compounds or structures which are mainly found in the epithelial cells, such as, for example, cytokeratins (Moll et al., Cell, 31, 11-24, 1982),

Various immortalized prostate cancer cell lines are in existence, including androgen-responsive LNCaP cancer cells, androgen-unresponsive DU-145 cancer cells, and androgen-unresponsive PC-3 cancer cells. Non-cancerous human prostate cell lines are also available, and may be used conventionally to analyze potential toxicity of drugs.

DU-145 is a cell line originally derived from a brain metastasis of a human prostate adenocarcinoma that retains the androgen independence of the original tumor and does not express a functional AR. This cell line has both LHRH-R and epidermal growth factor receptors (“EGFR”), and produces the EGFR ligands, transforming growth factor a (TGF-α) and EGF. The androgen-unresponsive PC-3 cell line is derived from a human bone metastasis of a grade IV prostatic adenocarcinoma, while the androgen-responsive LNCaP cell line is derived from a human lymph node metastasis. None of these are derived from primary tumor epithelial cells.

Two African American prostate cancer-derived human prostate cancer cell lines, MDA PCa and E006AA, are described by Navone et al. (“Establishment of two human prostate cancer cell lines derived from a single bone metastasis.” Clin. Cancer Res. 3: 2493-2500, 1997.) and Koochekpour et al. (“Establishment and characterization of a primary androgen-responsive African-American prostate cancer cell line,” E006AA. Prostate 60: 141-152, 2004.). The MDA PCa cell line was derived from a single bone metastasis, and thus is not a primary tumor of prostate epithelial cells. The E006AA cell line was established from a patient with a clinically localized prostate cancer, but is not tumorigenic in nude mice. Thus, those cell lines are not good models for studying primary prostate cancer because they do not reflect accurately the in situ characteristics of malignant prostate epithelium.

Thus, there remains a need in the art for improved treatments for prostate cancer that are highly effective in causing disease remission and in preventing progression of the disease to more advanced and aggressive stages while still exhibiting low toxicity.

### SUMMARY OF THE INVENTION

In light of the above needs, it is an object of the present invention to identify improved methods for screening potential therapeutic agents for the treatment of prostate cancer.

Furthermore, it is an object of the present invention to provide new models for studying treatments for patients having prostate cancer, including hormone refractory prostate cancer.

Additionally, it is an object of one or more embodiments of the present invention to provide cell lines that may be used to screen efficacy of treatments against prostate cancer and screen toxicity of such treatments against normal healthy prostate tissue.

Further, it is an object of one or more embodiments of the present invention to provide immortalized primary African American prostate cancer cells that will accurately reflect the in situ characteristics of malignant epithelium of primary prostrate tumors and provide the ability to generate tumors in vivo.

Also, it is an object of one or more embodiments of the present invention to provide immortalized primary prostate cancer cells that are androgen-responsive, and thus will be useful to answer questions regarding therapeutics targeted at AR.

The various embodiments of the present invention achieve these and other objects by providing immortal human prostate cell lines, including at least one immortal human prostate cancer cell line derived from a sample of a epithelial prostate tissue excised from a prostate cancer primary tumor of a human patient. The prostate cancer cell line produces long-lasting human tumors when injected into a host animal, such as a mouse. The cell lines, which typically have a diploid karyotype and express function AR, do not require exogenous growth factors for growth in vitro. One such prostate cancer cell line, designated RC-77T/E, will be deposited if required under the Budapest Treaty or the rules of the U.S. Patent and Trademark Office with the American Type Culture Collection (12301 Parklawn Drive, Rockville Md. 20852).

Additional embodiments of the invention further include at least one immortal healthy human prostate tissue cell line derived from a sample of healthy prostate epithelial tissue excised from a human patient. Preferably, this immortal healthy human prostate tissue cell line is derived simultaneously from the same human patient as an immortal human prostate cancer cell line to enable comparative studies to be conducted between at least one immortal healthy human prostate cell line and at least one immortal human prostate cancer cell line having a high degree of genetic similarity. One such healthy human prostate cancer cell line, designated RC-77N/E, will be deposited if required under the Budapest Treaty or the rules of the U.S. Patent and Trademark Office with the American Type Culture Collection (12301 Parklawn Drive, Rockville Md. 20852). Cell line RC-77T/E and cell line RC-77N/E were derived from prostate tissue excised from the same human patient, an African American male.

Embodiments of the invention include immortalized cell lines of a pair of non-malignant and malignant tumors derived from an African American prostate cancer patient using HPV-16E6E7. These cell lines, designated RC-77N/E for the line derived from non-malignant tumor cells and RC-77T/E for the line derived from malignant tumor cells, have been successfully grown past passage 40. Both of these preferred cell lines exhibit epithelial morphology and are androgen sensitive. The RC-77T/E cells produced tumors when injected subdermally in SCID mice whereas the RC-77N/E cells produced no tumor in SCID mice. Both cell lines express androgen-regulated prostate-specific homobox gene NKX 3.1, epithelial cell specific cytokeratin 8, androgen receptor (“AR”), prostate specific antigen (“PSA”), and p16. Chromosomal analyses show that both cell lines are similar, having near diploid human male (XY) chromosome counts typically within the 45-48 range. Further, the RC-77T/E cell line has new marker chromosomes: M1B=del/t(4;?)(q28;?), M5=16q+ in addition to those observed in the RC-77N/E cell line (M1=del(4)(q28q34)+hsr in some, M1A=t(4q;?),M2=der(9?),M2A=del(M2p-),M3=iso(?), M4=der(22?)). These preferred cell lines comprise a novel establishment of a pair of non-malignant and malignant tumors derived from an African American prostate cancer patient, and individually and in tandem provide novel tools to study the molecular and genetic mechanisms of prostate carcinogenesis, especially for high-risk African American men.

Further embodiments of the invention include methods for testing antitumor and antimetastasis therapies that involve culturing cells of a cell line according to the invention, treating those cells with the antitumor or antimetastasis therapy (e.g., a test compound), and determining whether growth of the cells is inhibited. In such methods, the treated cells may be treated in vitro and then assessed, or treated in vitro and then injected into a host animal whereby inhibition is thereafter determined by assessing whether a tumor is formed or metastasis occurs. Alternatively, such methods may include injecting the cells into a host animal without any treatment, waiting for a tumor and/or metastasis to occur, treating the host animal with the antitumor or antimetastasis therapy, and then determining whether growth of tumor or metastasis is inhibited.

Test compounds may include, for example, peptides, proteins, non-peptide compounds, synthetic compounds, fermentation products, cell extracts, plant extracts, animal tissue extracts, and plasma; these compounds may be new compounds or commonly known compounds. Further, an immortalized malignant cell culture of the present invention can be treated with the test compound and compared with an intact control of an immortalized non-malignant cell culture to evaluate the therapeutic/preventive effects of the test compound with changes such as those in (1) proliferation rates of the cells, (2) expression of markers for malignancy and/or metastases, and/or (3) cell morphology linked with malignant tumor formation.

Being selected from among the test compounds described above by using the screening method of the present invention, a compound thus selected may be identified as a potential safe therapeutic or other pharmaceutical of low toxicity for prostate cancers. Furthermore, a compound derivatized from such aforementioned compounds selected by screening can also be used similarly.

A compound obtained by said screening method may have formed a salt. Said salt is exemplified by salts with physiologically acceptable acids (e.g., inorganic acids, organic acids), bases (e.g., alkali metals), etc., with preference given to physiologically acceptable acid adduct salts. Such salts include, for example, salts with inorganic acids (e.g., hydrochloric acid, phosphoric acid, hydrobromic acid, sulfuric acid) and salts with organic acids (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid).

A pharmaceutical containing a compound obtained by said screening method or a salt thereof can be produced by a commonly known production method or a method based thereon. The preparations thus obtained can be used with, for example, humans or mammals (e.g., rats, mice, guinea pigs, rabbits, sheep, swine, bovines, horses, cats, dogs, monkeys) because they are safe and of low toxicity.

Examples of dosage forms for the aforementioned preparations include, for example, tablets (including sugar-coated tablets and film-coated tablets), pills, capsules (including microcapsules), granules, fine subtilae, powders, syrups, emulsions, suspensions, injectable preparations, inhalants, and ointments. These preparations are prepared in accordance with commonly known methods.

Other embodiments of the present invention include methods for obtaining the cell lines according to the invention.

The various embodiments of the invention having thus been generally described, several illustrative embodiments will hereafter be discussed with particular reference to several attached drawings and in view of various experimental examples.

### STATEMENT REGARDING DEPOSIT

Cell line RC-77T/E and RC-77N/E will be deposited in the American Type Culture Collection (“ATCC”) in Rockville, Md., if required by the U.S. Patent and Trademark Office per 37 CFR § 1.809(a), during the pendency of this application. As necessary, the cell lines will be available to the public as of the issue date of a patent on this subject matter, will be replaced if the culture mutates or becomes nonviable, and will be maintained for a term of 30 years, or five years after the last request for such deposit, or for the effective life of the patent, whichever is longest.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A and FIG. 1B are black and white photographs of, respectively, cultures of RC-77N/E and RC-77T/E showing the typical epithelial morphology of those cell lines.

FIG. 2 is a black and white photograph showing a RT-PCR result with E6 primer for cells of lines RC-77N/E and RC-77T/E in comparison to controls.

FIG. 3 is a table reporting experimental results for the identified phenotypic properties of RC-77N/E and RC-77T/E prostate cancer cell lines.

FIG. 4 is a graph comparing experimental results for the androgen sensitivity of RC-77N/E and RC-77T/E cells.

FIG. 5A and FIG. 5B comprise color photographs depicting the three-dimensional structure formation of RC-77T/E cells.

FIG. 5C is a chart comparing experimental results for ability of the RC-77N/E and RC-77T/E cells to form organoids in a rotating wall vessel culture.

FIG. 6 is a color photograph depicting a poorly differentiated adenocarcinoma produced by inoculation of the RC-77T/E into SCID mice.

FIG. 7A and FIG. 7B are black and white photographs depicting karyotypes of RC-77N/E cells and RC-77T/E cells, respectively.

FIG. 8 is a table reporting experimental results for the identified karyological properties of the RC-77N/E and RC-77T/E prostate cancer cell lines.

### DETAILED DESCRIPTION OF THE PREFERRED EMBODIMENTS

The term “cell line,” as used herein, refers to individual cells, harvested cells, and cultures containing the cells, so long as they are derived from cells of the cell line referred to. A cell line is said to be “continuous,” “immortal,” or “stable” if the line remains viable over a prolonged time, typically at least about six months. To be considered a cell line, as used herein, the cells must remain viable for at least 40 passages in the absence of exogenous growth factors. A “cell strain,” in contrast, refers to cells that do not remain viable over a prolonged time in the absence of exogenous growth factors.

A cell line is said to be “malignant” if, when the cell line is injected into a host animal, the host animal develops tumors or cancers that are anaplastic, invasive, and/or metastatic. A “human” tumor is comprised of cells that have human chromosomes. Such tumors include those in a human patient, and tumors resulting from the introduction of a human malignant cell line into a non-human host animal if cells from such tumors have human chromosomes. A tumor is said to be “long-lasting” when the tumor persists in an animal for at least about one month.

Tissue or a cell line are “normal cells” or “healthy” if they are not pre-cancerous, cancerous or derived from cancerous tissue.

A cell is said to be “diploid” if it contains two complete or substantially complete sets of paired chromosomes. A human diploid cell will have about 46 chromosomes, two times the normal haploid number.

“Growth factors” can include one or more of Oncostatin M, tumor necrosis factor, interleukin-1, interleukin-2, interleukin-2, and the HIV-1 transactivator TAT. Several growth factors have been identified in HTLV-II-conditioned medium (HTLV-II CM), obtained by growing HTLV-II-infected CD4⁺ T lymphocytes. One of these factors, oncostatin-M (Zarling et al. (1986) Proc. Natl. Acad. Sci. USA, 83: 9739-9743; Nair et al. (1992) Science 255: 1430-1432), is a 30 kD growth regulator originally identified by its ability to inhibit the growth of A375 melanoma cells and other human tumor cell lines, and to stimulate the proliferation of normal human fibroblasts and endothelial cells (Miles et al. (1992) Science 255: 1432-1434; Brown, T. J. (1987) J. Immunol. 139: 2977-2983).

The growth of a cell line is said to be “inhibited” if, when assayed by means such as radioisotope incorporation into the cells, the treated cells proliferate at a rate that is less than about 80% of the proliferation rate of untreated control cells, and preferably less than about 70% of the untreated cell proliferation rate.

As used herein, an “effective amount” of a composition is an amount sufficient to kill the targeted cells in a background population of non-targeted cells. Where appropriate in context, a “pharmaceutically effective amount” of a composition is an amount that is sufficient to kill the targeted cells, inhibit proliferation of the targeted cells, or initiate remission of a targeted cancer when that amount is administered to a stricken animal as a pharmaceutical formulation.

Unless defined otherwise, all technical and scientific terms used herein have the same meaning as commonly understood by one of ordinary skill in the art to which this invention belongs. Although any methods and materials similar or equivalent to those described can be used in the practice or testing of the present invention, the preferred methods and materials are now described. All publications and patent documents referenced in this application are incorporated herein by reference.

One set of paired malignant and non-malignant immortalized prostate cell lines according to a preferred embodiment of the invention will now be discussed in detail. Primary cell cultures were created by first obtaining malignant tumor tissue (“RC-77T”) and non-malignant tissue (“RC-77N”) under permission from a 63-year old African American patient diagnosed with prostate cancer. A radical prostatectomy was performed by the patient's physician according to Water Reed Army Medical Center and the Uniformed Services University of the Health Sciences Internal Review Board Protocol. Pathology confirmed that the patient had clinical stage T3c adenocarcinoma with poor differentiation (Gleason 7). Non-malignant cells RC-77N were obtained from non-cancerous regions of the removed prostate while malignant cells TC-77T were obtained from the cancerous regions, as confirmed by histopathological examination.

The procedure for generating primary prostate cell cultures from cell samples RC-77N and RC-77T utilized herein was done as generally described by Yasunaga et al. (“A novel human cell culture model for the study of familial prostate cancer.” Cancer Res. 61: 5969-5973, 2001.). The tumor and non-malignant tissue obtained by an experienced pathologist was chopped into small fragments, 1-2 mm in size with a sterile blade. The resulting small cell clumps were placed into several type 1 collagen-treated dishes (obtained from Becton-Dickinson, Boston, Mass.) containing growth medium and were allowed to attach for a week to the bottom surface of the culture dishes. The cells were incubated at 37° C. in a humidified air of 5% CO₂ until reaching semi-confluency. Aliquots of the primary cultures were then frozen and stored in liquid nitrogen until the cells were re-established in secondary culture for additional serial passages. For serial passages, routine trypsinization was used once a week in the collagen-treated culture dishes, and the split ratio of the cells was 1-2. Keratinocyte serum-free medium (“K-SFM”) supplemented with bovine pituitary extract and recombinant epidermal growth factor (obtained from Life Technologies, Inc., Gaithersburg, Md.) was used for growing and maintaining the cells.

At passage 4, the actively proliferating RC-77T and RC-77N cells grown in K-SFM with supplements were infected with a recombinant retroviral construct, LXSN-HPV16E6E7 (obtained from Dr D. A. Galloway, Seattle, Wash.) containing the E6 and E7 genes of HPV-16 and a neomycin resistance gene, as described by Galloway et al. (“Human papillomaviruses and carcinomas.” Adv. Virus Res. 37: 125-171, 1989.). Samples of the grown RC-77T and RC-77N cells were transduced through infection using polybrene at the concentration of 10 μg/ml and incubated at 37° C. in 5% CO2 overnight. The infected cells were then washed with phosphate buffered saline (“PBS”), then incubated and subcultured weekly for further serial passages. Selection by G418 agent was not was necessary because the uninfected RC-77T and RC-77N cells senesced at passage 6. The remaining cells from RC-77T were designated RC-77T/E and the remaining cells from RC-77N were designated RC-77N/E.

To determine whether the human prostate cancer cell lines became immortalized by the expression of HPV-16E6E7, a retrovirus construct expressing HPV-16E6E7 was introduced into passage=3 RC-77N/E and RC-77T/E cells through overnight infection. Non-infected cells could not be propagated serially beyond 5 subcultures. In contrast, the HPV-16E6E7- infected RC-77N/E and RC-77T/E cells were found to have an apparently unlimited lifespan. Lines RC-77N/E and RC-77T/E and since been successfully subcultivated for greater than 40 passages over the course of 1 year with no evidence of decreased proliferation capacity.

### Experiment 1

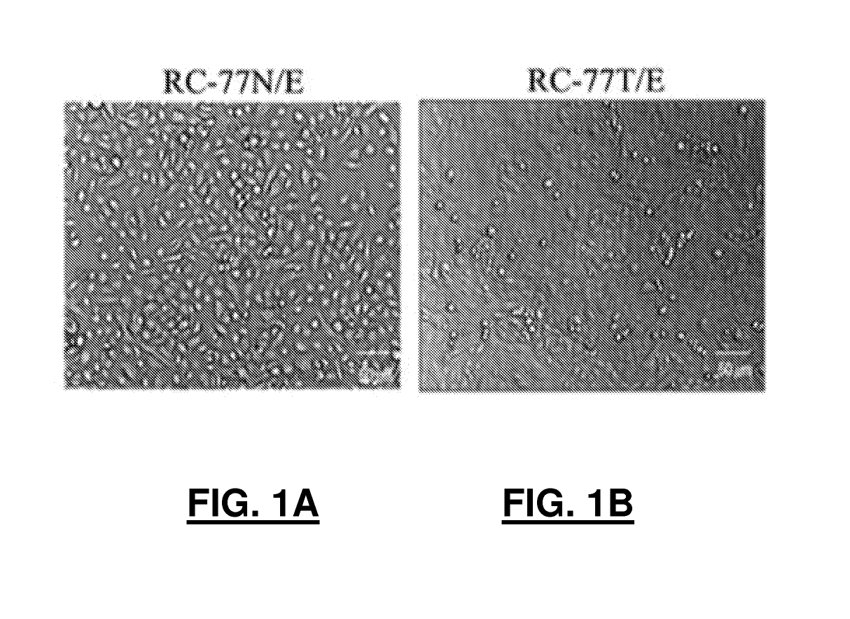

The morphological characteristics of the RC-77T/E and RC-77N/E cell lines were studied via microscope. As depicted by the comparative black and white photographs of FIG. 1A and FIG. 1B. the RC-77N/E and RC-77T/E cells, respectively, exhibit typical epithelial morphology, with the cells growing as adherent cells with some being more piled-up on each other in certain areas.

### Experiment 2

In order to genetically characterize the two newly created cell lines RC-77T/E and RC-77N/E, a RT-PCR assay was done according to the procedure described by Xu et al. (“Expression profile of an androgen regulated prostate specific homobox gene NKX3.1 in primary prostate cancer.” J Urol 163: 972-979, 2000.). Total RNAs from culture cells were extracted with RNAzol B (obtained from TEL-TEST Inc., Friedswood, Tex.) according to the manufacturer's protocol and quantified with a Nucleic Acid Quantitation Kit (obtained from NBI, Plymouth, Minn.). Total RNA (1 μg) was reverse transcribed into cDNA with an RNA PCR kit (obtained from Perkin-Elmer, Foster, Calif.), and 1/10 of the reverse-transcribed product from each sample was used for PCR to amplify AR, NKX3.1, CK8, and HPV-16E6 genes respectively. The expression of CK8 was used as an internal control for input RNA as well as the marker for epithelial cells. To verify the validity of CK8 as the internal control, CK8 was compared with the house-keeping gene, GAPDH, in the same cDNA samples. The condition of PCR for the individual gene was optimized to analyze the amplified product in the linear range of amplification by adjusting amplification cycles for each set of primers.

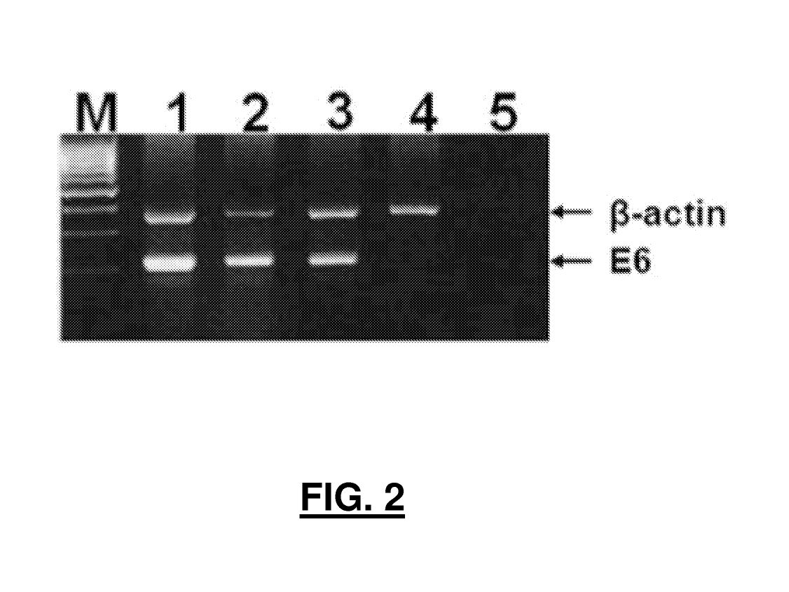

To confirm that the immortalized RC-77N/E and RC-77T/E contain the transduced HPV-16E6E7 gene, RT-PCR was carried out with 1532T cells as positive control and DU145 cells as negative control. To verify the integrated E6 and E7 genes of HPV-16 were present in the RC-77T/E and RC-77N/E cell lines, RT-PCR with E6 primer was carried out. FIG. 2 provides a photograph of this assay. As shown in FIG. 2, strong expression of E6 gene (210 bp) was detected for both RC-77T/E cells, passage 25 (lane 1 of FIG. 2), and RC-77N/E, passage 25 (lane 2 of FIG. 2). The 1532T cells, which served as a positive control are in lane 3 of FIG. 2 while the DU145 cells, serving as a negative control, were in lane 4 of FIG. 2. As one would expect with a positive immortalization of the cells, the expression of HPV-16E6 gene was detected in both the RC-77T/E and RC-77N/E cell lines and the positive control 1532T cells but not the negative DU-145 cells. In FIG. 2 it also is visible that expression of β-actin gene (315 bp) was present in both the RC-77T/E and RC-77N/E lines, monitored as an internal positive PCR control and with H₂O as negative control (lane 5).

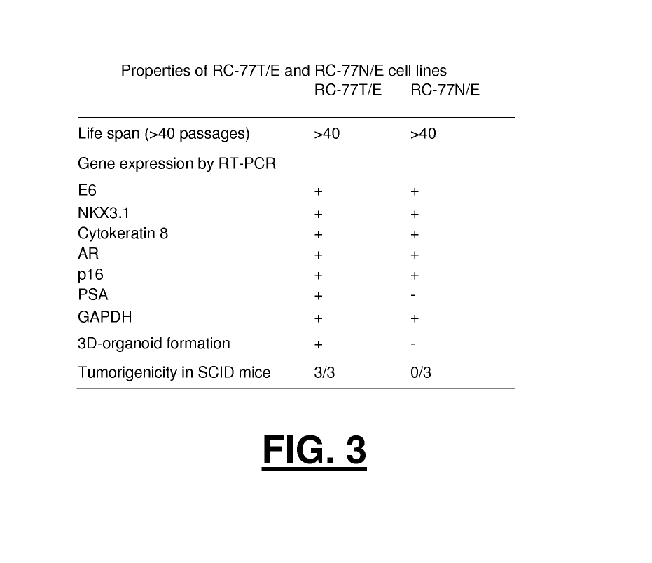

RC-77T/E and RC-77N/E cells were further analyzed in similar fashion to determine the expression of other specific markers by RT-PCR. The results of these PCR analyses are summarized in the table reproduced as FIG. 3, which identifies the various markers expressed in one or both cell lines after 40 passages. As shown in this table, along with E6, at greater than 40 passages Androgen-regulated prostate specific homobox gene NKX3.1, epithelial cell-specific cytokeratin 8, AR, and p16 were expressed in both cell lines. Additionally, PSA expression in only the malignant cell line RC-77T/E was determined by real-time PCR and immunofluorescence. The primer sequences and the expected size of PCR products were consistent with as was reported by Xu et al.

### Experiment 3

Since androgen receptor expression was observed both cell lines, an experiment was conducted to determine the effects of androgen stimulation on the growth of RC-77N/E and RC-77T/E cells. Cells were grown in serum-free K-SFM at different doses of Methyltrienolone (R1881), a synthetic androgen, for 4 days.

In this experiment, 2×10⁴ cells per well of each line were grown in serum-free K-SFM with 0.1% BSA in the presence of 0, 0.1, 1.0, 10.0 and 100.0 nM for 4 days. K-SFM was supplemented with or without Methyltrienolone (R1881) (obtained from Perkin-Elmer, Waltham, Mass.), at concentrations of 0.1, 1, 10 and 100 nM, respectively. Cell proliferation was determined by MTT assay according to a procedure as generally described by Wells et al. (“Luteinizing hormone-releasing hormone agonist limits DU-145 prostate cancer growth by attenuating epidermal growth factor receptor signaling.” Clin. Cancer Res. 8: 1251-1257, 2002.).

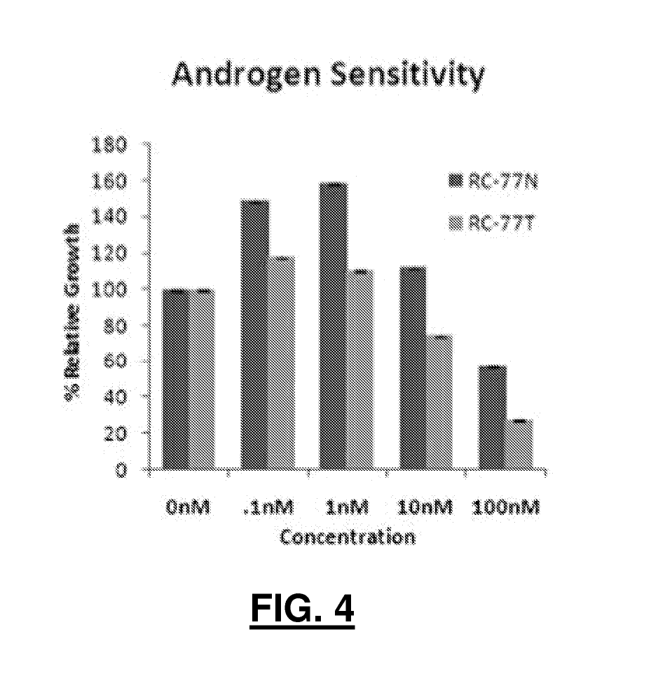

The results from this experiment are illustrated in the chart of FIG. 4, with percent growth being determined by the number of cell counts being expressed as the mean value of triplicate observation. Significant differences in cell growth between RC-77T/E cells and RC-77N/E cells was observed at 1.0 nM R1881 in stimulation. Further, the RC-77T/E cells were found responsive to 0.1 nM R1881, while RC-77N/E cells were only responsive at the higher dosage of 1.0 nM. Significant stimulation in cell growth was observed for RC-77T/E cells and RC-77N/E cells at 1.0 nM R1881. However, the higher dosage of 100.0 nM R1881 resulted inhibition of cell growth for both cell lines.

### Experiment 4

A three-dimensional culture of prostate cells in rotating wall vessel (“RWV”) experiment was conducted to assess the tumorigenicities of RC-77T/E and RC-77N/E. The formation of such non-adherent three dimensional organoids can serve as a criterion for in vitro tumor forming potential. In order to determine whether cells from the two lines demonstrated this characteristic, three-dimensional RWV conditions were established according to the procedures set forth previously by Sung et al. (“Coevolution of prostate cancer and bone stroma in three-dimensional coculture: implications for cancer growth and metastasis.” Cancer Res. 68: 9996-10003, 2008.). In this experiment, 2×10⁷ of both RC-77N/E and RC-77T/E cells were seeded in RWV systems in serum-free KGM media and stopped, and the prostate organoids and medium was collected on respective days.

The extent of three-dimensional structure formation was determined by measuring the disappearance of single cells using the Coulter Counter Z1. The index of the degree of individual cells incorporated into organoids was measured utilizing the formula 100× (N0/ND), where N0 is the total cell number input and ND is the total number of particles/cells after respective days of incubation as determined by counting in a Coulter Counter Z1 (see Yates et al.: “Luteinising hormone-releasing hormone analogue reverses the cell adhesion profile of EGFR overexpressing DU-145 human prostate carcinoma subline.” Br. J. Cancer 92: 366-375, 2005.). Organoids were harvested, and fixed with 1% formaldehyde and sectioned for histopathology. Images were thereafter taken with a DSU Confocal Unit (Olympus) and images were processed with Metamorph software.

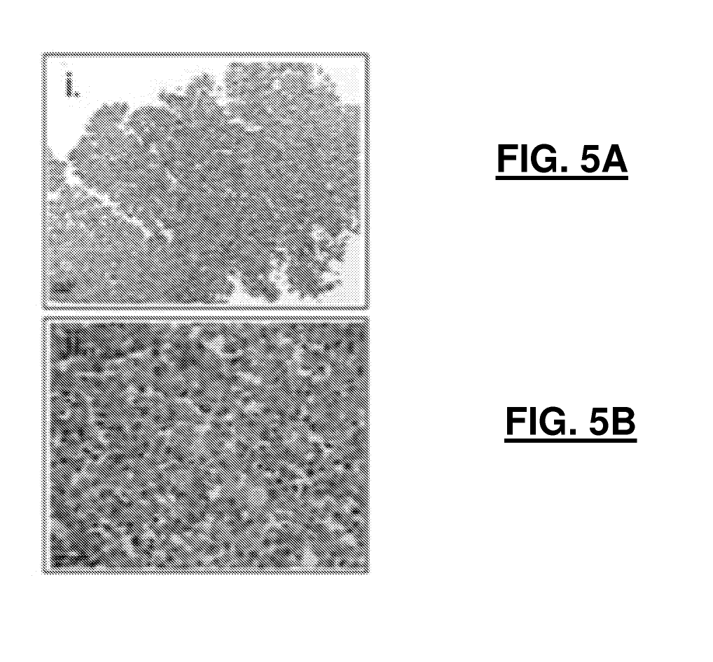

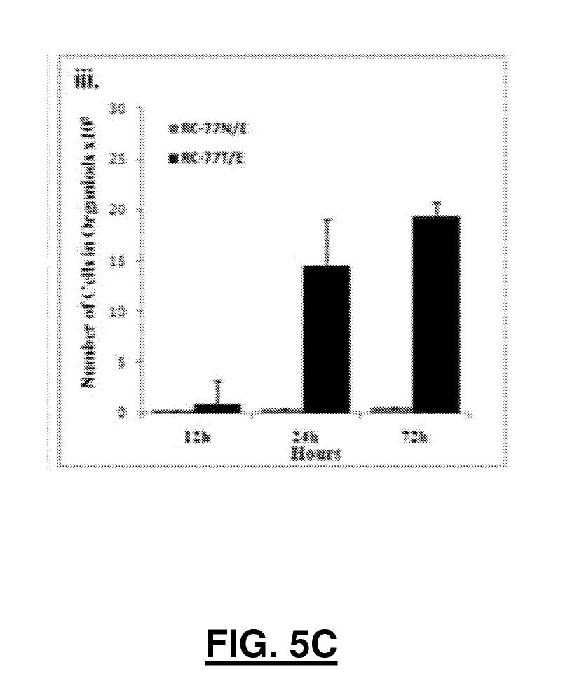

The RC-77T/E malignant-derived cells formed three dimensional tissue structures over a 5-day period, while conversely the RC-77N/E cells remained suspended as single cells and did exhibit any significant observable organoid formation. Histopathological analysis of the RC-77T/E organoids showed tumor tissue-like structures, as shown in the images of FIG. 5A and FIG. 5B. Vertically, all RC-77T/E cells where cohesive, as measured by number of suspended cells after each time interval.

The average number of cells in an organoid formed by RC-77T/E cells was significantly higher than those formed by RC-77N/E cells, with maximal incorporation of individual RC-77T/E cells into organoids occurring at day 3 of RWV culture as reported in the chart reproduced in FIG. 5C.

### Experiment 5

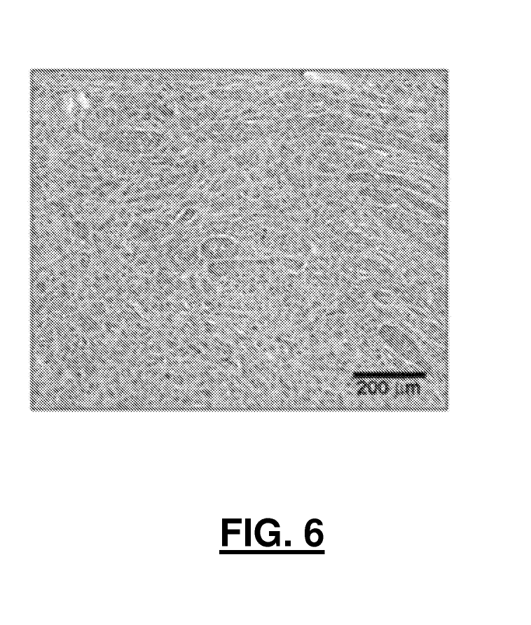

To determine tumorigenicity in vivo for the RC-77T/E (passage 45) and RC-77N/E (passage 45) cell lines, 1×10⁷ cells were injected subcutaneously into SCID mice (3 mice for each cell line). The mice were observed for 6 months for tumor development. All the animals inoculated with RC-77T/E cells developed tumors within 4.5 months at the site of inoculation and the tumors reached 10 mm size at 6 months. Microscopic examination of sections of these tumors revealed poorly differentiated adenocarcinoma. FIG. 6 is a pathology photograph of showing the highly atypical poorly differentiated adenocarcinoma produced by inoculation of the RC-77T/E cell line into SCID mice, with cohesive groups of malignant cells extensively infiltrating the surrounding tissues.

Conversely, no tumor formation was detected in animals inoculated with RC-77N/E cells for the 6-month observation period. Thus, as reflected in the table of FIG. 3, it was determined that RC-77T/E exhibited tumorigenicity in SCID mice while RC-77N/E did not.

### Experiment 6

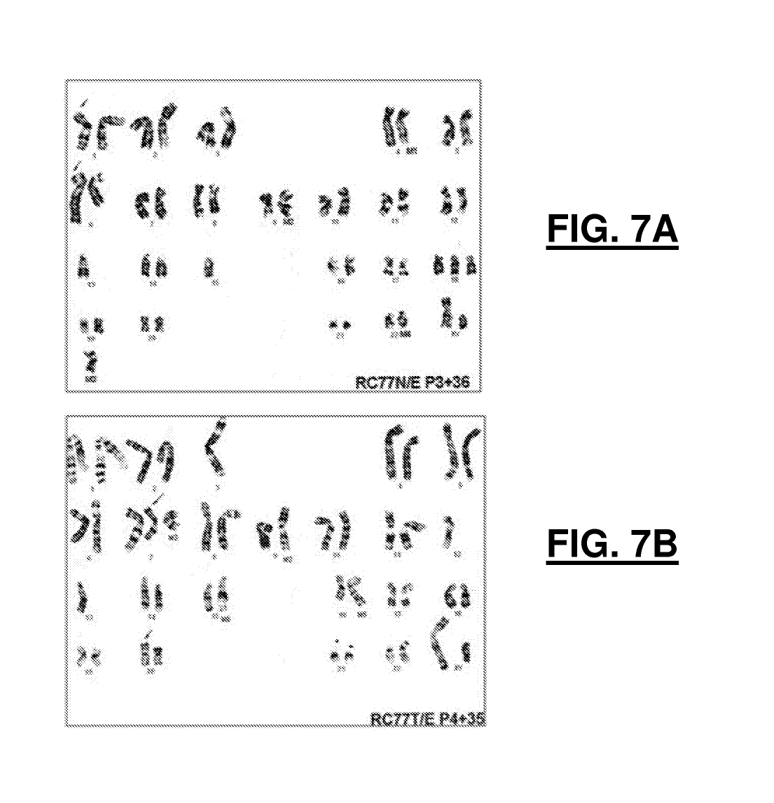

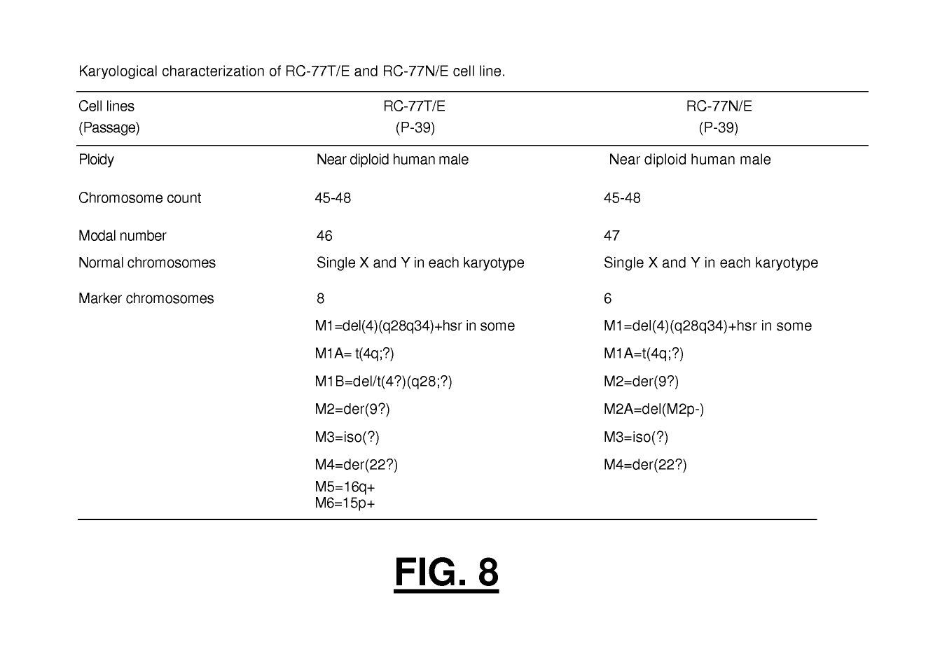

For this experiment, chromosome studies were performed at passage 39 of cell lines RC-77T/E and RC-77N/E, including chromosome counts, ploidy distribution, and Giemsa (G)-banded karyotypes as performed by standard protocols (see Hukku et al.: “Role of chromosome 5 in immortalization and tumorigenesis of human keratinocytes.” Cancer Genet. Cytogenet. 68: 22-31, 1993; and see International System for Human Cytogenetic Nomenclature. In: An International System for Human Cytogenetic Nomenclature. Mitelman F (ed). S. Karger, Basel, 1995.). FIG. 7A is a photograph depicting a karyotype of RC-77N/E (passage 39) cells, while FIG. 7B is a photograph depicting a karyotype of RC-77T/E (passage 39) cells. Further, the karyological characteristics of RC-77T/E and RC-77N/E are summarized for comparison in the table reproduced as FIG. 8.

This experiment confirmed that both cell lines are similar, having near diploid human male (XY) with most cells having chromosome counts in the 45-48 range. As reported in FIG. 8, the modal number of the RC-77T/E cell line is 48 whereas the modal number of RC-77N/E cell line is 46. Four marker chromosomes were detected in RC-77N/E cell line whereas eight marker chromosomes were detected in RC-77T/E cell line. Single X and Y in each karyotype of both cell lines is observed, as can be seen from inspection of FIG. 7A and FIG. 7B.

The eight marker chromosomes found in RC-77T/E cell line are as listed in FIG. 8. Compared to RC-77N/E cell line, the RC-77T/E cell line shows new changes in marker M1 as M1B and one copy of normal chromosomes 15 and 16 as marker M6 and M5, gaining p+ and q+ extra material, respectively. Only six marker chromosomes were found in the RC-77N/E cell line, including those found the same marker chromosomes (M1=del(4)(q28q34)+hsr in some, M1A=t(4q;?), M2=der (9?), M2A=del(M2p-), M3=isoz(?) and M4=der(22?) detected in the RC-77T/E cell line.

Both cell lines are cytogenetically similar, near diploid human male (XY). However, RC-77T/E cell line has new marker chromosomes, (M1B=del/t(4?)(q28;?), M5=16q+and M6=15p+) in addition to those observed in the RC77N/E cell line (M1=del(4)(q28q34)+hsr in some, M1A=t(4Q;?), M2=der(9?), M2A=del(M2p-), M3=iso(?), M4=der(22?).

The 4q alteration observed in both cell lines is also an observed chromosome change in a prior established human prostate cancer cell line derived from primary tumor of a familial prostate cancer patient (see Yasunaga et al., supra), providing possible evidence of a prostate cancer susceptibility locus on chromosome 4q (see, Smith et al.: “Major susceptibility locus for prostate cancer on chromosome 1 suggested by a genome-wide search.” Science 274: 1371-1374, 1996.). Additionally, the marker chromosome 15+ has been observed in a tumorigenic primary tumor-derived human prostate cancer cell line (see Ko et al.: “A novel neoplastic primary tumor-derived human prostate epithelial cell line.” Int. J. Oncol. 22: 1311-1317, 2003.) while the marker chromosome 16q is the most frequent region alteration observed in primary prostate cancer (see Carter et al.: “Epidemiologic evidence regarding predisposing factors to prostate cancer.” Prostate 16: 187-197, 1990; and see Kunimi et al.: “Allelotyping of human prostatic adenocarcinoma.” Genomics 11: 530-536, 1991.). Furthermore, some of the alteration of chromosomes observed in the RC-77T/E and RC77N/E cell lines have been reported in prior cell lines. The presence of a marker chromosome involving chromosome 4 (M1=del(4)(q28q34)+hsr in some) in both cell lines has been reported in other African American derived metastatic prostate cancer cell line (MDA PCa) and a primary African American prostate cancer cell line, E006AA (see Navone et al., supra, and Koochekpour et al., supra.).

It is understood that the examples and embodiments described herein are for illustrative purposes only and that various modifications or changes in light thereof will be suggested to persons skilled in the art and are to be included within the spirit and purview of this application and scope of the appended claims.

Having described preferred embodiments of the invention, it will now become apparent to those of ordinary skill in the art that other embodiments incorporating these concepts may be used. Accordingly, it is submitted that that the invention should not be limited to the described embodiments but rather should be limited only by the spirit and scope of the appended claims.

Thus, although the invention has been described and illustrated with a certain degree of particularity, it is understood that the present disclosure has been made only by way of example, and that numerous changes in the combination and arrangement of steps, ingredients, or processes can be resorted to by those skilled in the art without departing from the spirit and scope of the invention, as will be claimed.

1. A genetically modified human malignant prostate epithelial cell which can be maintained as a stable, substantially homogeneous cell line in-vitro, said genetically modified cell comprising: a malignant prostate epithelial cell of human origin which is stable when maintained in culture and which (i) has androgen dependence; (ii) produces substantially homogenous progeny continuously while maintained in culture; and (iii) causes tumors when introduced with a sufficient amount of like cells into a non-human mammalian host animal.

2. The genetically modified human malignant prostate epithelial cell as recited in claim 1, wherein the cell expresses androgen receptor.

3. The genetically modified human malignant prostate epithelial cell as recited in claim 1, wherein said cell expresses at least one active substance selected from the group consisting of a cytokeratin.

4. The genetically modified human malignant prostate epithelial cell as recited in claim 1, wherein said cell is diploid male.

5. The genetically modified human malignant prostate epithelial cell as recited in claim 1, wherein said cell is derived from tissue of a primary prostate cancer tumor.

6. The genetically modified human malignant prostate epithelial cell as recited in claim 1, wherein said cell is derived from a tissue sample taken from an African American.

7. A human prostate cell line containing genetically modified human malignant prostate epithelial cells according to claim 1, wherein the cell line is RC-77T/E.

8. A laboratory animal comprising a tumor formed from implantation of a cell of claim 1.

9. The laboratory animal of claim 3, wherein the laboratory animal is a mammal.

10. The laboratory animal of claim 4, wherein the mammal is a mouse.

11. A composition of matter consisting essentially of a plurality of cells of claim 1 and a growth media therefore.

12. A process for assaying the prostate cancer-inhibiting activity of drug agents comprising the steps of:
(a) forming a culture of cells of the RC-77T/E cell line of claim 2 in a culture medium;
(b) exposing said culture to an amount of a test agent;
(c) incubating the culture exposed to said test agent for a suitable period of time, and
(d) counting the number of viable RC-77T/E cells remaining after completion of the incubation period, wherein the number of viable RC-77T/E cells is used as an indication of the prostate cancer-inhibiting activity of the test agent.

13. The process according to claim 12, further comprising the steps of:
(e) forming a second culture of cells of the RC-77N/E cell line in a culture medium, wherein the cell line is RC-55N/E;
(f) exposing said second culture to an amount of said test agent;
(g) incubating the second culture exposed to said test agent for said suitable period of time, and
(h) counting the number of viable RC-77N/E cells remaining after completion of the incubation period, wherein the number of viable RC-77N/E cells is used as an indication of the toxicity of the test agent.

14. The process according to claim 13, further comprising repeating said steps (a) through (h) for different test agents and using said indications of activity and indications of toxicity to select drug candidates for additional study.

15. The process according to claim 14, wherein said additional study comprises in vivo studies.

16. The process according to claim 7, further comprising repeating said steps (a) through (d) for different test agents and using said indications of activity to select drug candidates for additional study.

17. The process according to claim 16, wherein said additional study comprises in vivo experiments.

18. A kit for studying prostate cancer, comprising a first culture of cells from the cell line of claim 2, and a second culture of cells from the cell line is RC-77N/E.

19. A method for studying prostate cancer, said method comprising the steps of:
(a) obtaining paired immortalized human prostate cell lines from tissue taken from the same human patient, said paired cell lines including a first and a second cell line, said first cell line comprising an immortalized human prostate cancer cell line derived from a sample of malignant epithelial prostate tissue excised from a prostate cancer primary tumor of said human patient, said second cell line comprising an immortalized human prostate tissue cell line derived from a sample of normal non-malignant prostate epithelial tissue excised from said human patient, wherein said first immortalized human prostate cancer cell line produces long-lasting human tumors when injected into a host non-human animal,
(b) forming test cultures of cells of the cell lines in a culture medium;
(c) exposing said test cultures to an amount of a test agent;
(d) incubating said test cultures exposed to said test agent for a suitable period of time, and
(e) monitoring changes to said cultures following said exposing relative to changes in control cultures.

20. The method according to claim 19, wherein said step of monitoring changes comprises for at least one of said test cultures counting numbers of viable cells from the first cell line before and after the incubation period, and comparing differences in the numbers of cells before and after the incubation period relative to said control cultures as an indication of prostate cancer-inhibiting activity of the test agent.

21. The method according to claim 19, wherein said step of monitoring changes comprises for at least one of said test cultures counting numbers of viable cells from the second cell line before and after the incubation period, and comparing differences in the numbers of cells before and after the incubation period relative to said control cultures as an indication of toxicity of the test agent to non-malignant prostate tissue.

22. The method according to claim 19, wherein said step of monitoring changes comprises for at least one of said test cultures examining cell morphology of cells before and after the incubation period, and comparing differences in said cell morphology before and after the incubation period relative to control cultures.

23. The method according to claim 19, wherein said step of monitoring changes comprises for at least one of said test cultures measuring expression of one or more markers for malignancy, metastases, or both.

24. The method according to claim 19, wherein said first and said second cell lines both comprise cells that have a diploid karyotype.

25. The method according to claim 19, wherein said first and said second cell lines both comprise cells that express functional androgen receptor.

26. The method according to claim 19, wherein said first and said second cell lines both comprise cells that do not require exogenous growth factors for growth in vitro.

27. The method according to claim 19, wherein said first cell line is RC-77T/E, and wherein said second cell line is RC-77N/E.
